(19) United States
(12) Patent Application Publication
(10) Pub. No.: US 2008/0260853 A1
(43) Pub. Date: Oct. 23, 2008

(54) EXTRACELLULAR MATRIX AS SURGICAL ADJUNCT IN A LUMPECTOMY PROCEDURE

(76) Inventor: LEIGH H. FIRESTONE, Berkeley, CA (US)

Correspondence Address: LEIGH HUNT FIRESTONE, 7100 NORFOLK ROAD, BERKELEY, CA, 94705, US

(21) Appl. No.: 11/876,963

(22) Filed: Oct. 23, 2007

Related U.S. Application Data
(63) Continuation-in-part of application No. 11/708,231, filed on Feb. 20, 2007.
Application No. 11/708,231 is a continuation-in-part of application No. 11/865,023, filed on Sep. 30, 2007.
(60) Provisional application No. 60/775,913, filed on Feb. 22, 2006.

(30) Foreign Application Priority Data
Feb. 21, 2007 (US) PCT/US2007/004332

(51) Int. Cl. A61K 35/38; A61K 35/407

(52) U.S. Cl. 424/553; 424/551

(57) ABSTRACT

The invention an article or composition of mammalian extracellular matrix (ECM) for placement in a lumpectomy space of a breast after tumor removal. The extracellular matrix article or composition can regenerate lost breast tissue, reduce the formation of scar tissue at the site of excision, and reduce a likelihood of local tumor recurrence at the lumpectomy site. The article can be sheet ECM, and the composition can be particulate ECM or emulsion or gel ECM.

### CROSS-REFERENCES TO RELATED APPLICATIONS

The present application is a continuation in part to application U.S. Ser. No. 11/708,231 filed Feb. 20, 2007 which draws priority from U.S. provisional application 60/775,913 filed Feb. 22, 2006. The present application also claims priority from PCT application PCT/US2007/004332 filed Feb. 21, 2007. The present application is also a continuation in part of U.S. Ser. No. 11/865,023 filed Sep. 30, 2007.

### FIELD OF THE INVENTION

The invention relates to devices for use in breast lumpectomy procedures.

### BACKGROUND OF THE INVENTION

Each year about 1 million women worldwide are diagnosed with breast cancer. Of these a large percentage undergo a lumpectomy procedure which entails surgical access to the breast from the skin surface to the identified lump and removal of the lump and surrounding tissue.

The most desirable outcome in a lumpectomy procedure is removal of the lump with what are termed clean margins. Clean margins mean that the cells that exist in the outer limits of the tissue removed from the breast do not indicate the presence of cancer or tumor tissue. The removed tissue is analyzed for whether it contains healthy cells surrounding the tumor (a clean margin) which indicates that the entire cancerous lump has been removed.

Typically after lumpectomy, the patient undergoes a series of radiation treatments to forestall tumor recurrence at the site. Radiation often causes scarring and makes recovery from the surgical procedure more difficult.

Recurrence of local tumors occur in about 10% of lumpectomy patients who receive radiation, and in about 20% of patients who do not, for whatever reason, elect to receive radiation. With high risk women who are usually young, and have aggressive tumors, local tumor recurrence will occur within the first 3 years after their lumpectomy procedure, and tumor recurrence rates are higher in high risk women—somewhere between 30 and 50% in the 5 years after surgery.

It would be desirable to develop a means and identify a material to assist in performing lumpectomy procedures that reduced the likelihood of local tumor recurrence.

### SUMMARY OF THE INVENTION

The invention is directed to an article or composition for placing at a site in a breast after tumor excision comprising mammalian extracellular matrix (ECM). The ECM can be small intestine submucosa, liver basement membrane, urinary bladder submucosa, or stomach submucosa. The mammalian ECM can be porcine. The ECM can be in a single sheet form, a particulate, or an emulsion or gel.

The invention also provides a method of regenerating excised tissue in a breast after a lumpectomy procedure comprising placing an article or composition comprising mammalian extracellular matrix (ECM) at a site of surgical excision of a tumor from said breast, and closing said surgical excision. The ECM can be porcine. The ECM can be small intestine submucosa, stomach submucosa, liver basement membrane, or urinary bladder submucosa.

The invention is also directed to a method of reducing a likelihood of local tumor recurrence in a breast after a lumpectomy procedure comprising placing an article or composition comprising mammalian extracellular matrix (ECM) at a site of surgical excision of a tumor from said breast, and closing said surgical excision. The ECM can be porcine. The ECM can be small intestine submucosa, stomach submucosa, liver basement membrane, or urinary bladder submucosa.

The invention also includes a method of reducing scar tissue formation in a breast after a lumpectomy procedure comprising placing an article or composition comprising mammalian extracellular matrix (ECM) at a site of surgical excision of a tumor from said breast, and closing said surgical excision. The ECM can be porcine. The ECM can be small intestine submucosa, stomach submucosa, liver basement membrane, or urinary bladder submucosa.

### BRIEF DESCRIPTION OF THE DRAWINGS

FIG. 1 depicts a cross sectional view of a breast having a tumor.

FIG. 2 depicts a cross sectional view of a breast after a lumpectomy procedure removing a tumor.

FIG. 3 depicts a cross sectional view of a breast after the space left after tumor removal is filled with small pieces of crumpled sheet extracellular matrix.

FIG. 4 depicts a cross sectional view of a breast after the space left after tumor removal is filled with either particulate extracellular matrix or emulsion or gel extracellular matrix.

### DETAILED DESCRIPTION OF THE INVENTION

The invention is an article or composition comprising mammalian extracellular matrix (ECM) for placing in the space remaining in a breast after a lumpectomy procedure in which a tumor and tissue next to the tumor are excised. The article can be a sheet or several sheets of ECM. The sheets can be placed in the excised tumor space, crumpled into balls, or wads and placed in the space in the breast, or rolled loosely or tightly and configured to fit into the space left after the tissue and tumor have been excised. The composition is particulate ECM or emulsion or gel ECM.

Accordingly, the particulate, emulsion or gel compositions can comprise ECM and other materials as well. Optimally, the composition comprises only ECM, and the ECM been processed so as to retain key growth factors and other molecules and proteins so that when the ECM is placed in the body, the ECM remodels to become the host tissue with which it is in contact. Other materials added to the ECM could be, for example, a therapeutic agent, a drug, added proteins or added cells.

Natural ECM materials suitable for use with the present invention include mammalian small intestine submucosa (SIS), stomach submucosa (SS), urinary bladder submucosa (UBS), dermis, or liver basement membranes (LBM) derived from sheep, bovine, porcine or any suitable mammal. Small intestine submucosa (SIS) is described in U.S. Pat. Nos. 4,902,508 (hereinafter the '508 patent), 4,956,178 (hereinafter the '178 patent) and 5,275,826; urinary bladder submucosa (UBS) is described in U.S. Pat. No. 5,554,389 (hereinafter the '389 patent), stomach submucosa (SS) is described in U.S. Pat. No. 6,099,567, and liver submucosa (LS) or liver basement membrane (LBM) is described in U.S. Pat. No. 6,379,710. See also U.S. Pat. No. 5,554,389, U.S. Pat. No. 4,902,508, and U.S. Pat. No. 5,281,422.

Although these particularly named extracellular matrices are known and have been isolated and used, there may be other mammalian tissues from which extracellular matrix can be isolated and prepared and as such would be suitable for the purposes of the invention. Extracellular matrix-like materials are also generally described in the article “From Cell-ECM Interactions to Tissue Engineering”, Rosso et al, Journal of Cellular Physiology 199, 174-180 (2004). Enamel matrices, which are the extracellular matrix in the tissue around forming teeth, are described in U.S. Pat. No. 7,033,611. The disclosures of all references cited herein are incorporated in their entirety by reference. Extracellular matrices from these tissues have been isolated, processed to retain key growth factors and structural molecules, and dried to become solid materials (sheets and particulates). Particulate forms can be rehydrated in a suitable buffer to become fluidized or emulsion or gel forms. Presently, these extracellular matrix articles and compositions are used for tissue grafting, wound healing, and tissue regenerative purposes.

The invention proposes use of these ECM articles and compositions and materials and forms for placement in a breast in the space remaining after a lumpectomy procedure for the purpose of reducing a likelihood of tumor recurrence in the breast after a lumpectomy. The ECM articles and compositions and materials and forms are also proposed in order to regenerate the tissue lost from the breast during the tumor excision. In addition, the ECM articles and compositions and materials and forms are also proposed in order to reduce localized scarring that can result from the surgical wound created by the lumpectomy procedure.

Methods of use of the articles and compositions of the invention are also contemplated, for example as part of a routine lumpectomy procedure to excise a breast tumor, and replace the lumpectomy space in the breast with mammalian ECM in a sheet form (i.e. as an article) or as a particulate or emulsion or gel form (i.e. as a composition). Surgical excision of the tumor, and placement of the ECM in the space in the breast after the excision is followed by closing the surgical wound with the ECM article or composition in the breast. Tissue regeneration or wound healing occurs within about 3 to 6 months post lumpectomy.

Mammalian tissue sources are in general any tissue having an extracellular matrix that can be isolated from a mammal and de-cellularized. Thus for example, most mammalian organs are tissue sources. The tissue sources can be for example any mammalian tissue, including but not limited to the small intestine, large intestine, stomach, lung, liver, kidney, pancreas, placenta, heart, bladder, prostate, tissue surrounding growing tooth enamel, tissue surrounding growing bone, and any fetal tissue from any mammalian organ. The decellularization process is important as the material needs to be without its native cells, but the process of the removing the cells need not be so stringent as to remove key active growth factors that contribute to the material's usefulness in the human body. Processes for isolated extracellular matrix from tissues are known in the art, as are processes of decellularizing these matrices.

Extracellular matrix can be obtained from the tissues of mammals by processes such as described in U.S. Pat. No. 5,554,389, U.S. Pat. No. 4,902,508, and U.S. Pat. No. 5,281,422. For example, the urinary bladder submucosa is an extracellular matrix that has the tunica mucosa (which includes the transitional epithelial layer and the tunica propria), a submucosal layer, 3 layers of muscularis, and the adventitia (a loose connective tissue layer). This general configuration is true also for small intestine submucosa (SIS) and stomach submucosa (SS). Obtaining enamel matrices is described in U.S. Pat. No. 7,033,611. Enamel matrix is extracellular matrix existing near forming teeth.

Natural ECM materials include mammalian small intestine submucosa (SIS), stomach submucosa (SS), urinary bladder submucosa (UBS), dermis, or liver basement membranes (LBM) derived from sheep, bovine, porcine or any suitable mammal. Small intestine submucosa (SIS) is described in U.S. Pat. Nos. 4,902,508, 4,956,178 and 5,275,826; urinary bladder submucosa (UBS) is described in U.S. Pat. No. 5,554,389, stomach submucosa (SS) is described in U.S. Pat. No. 6,099,567, and liver submucosa (LS) or liver basement membrane (LBM) is described in U.S. Pat. No. 6,379,710. In the preparation process, native extracellular matrices are prepared so that their bioactivity is preserved, including many cellular and transcriptional and translational event. Assays for determining these activities are standard in the art.

Many of these ECM compositions are generally comprised of the same tissue layers and are prepared by the same method, the difference being that of the starting material (i.e. from one organ versus another). The matrices are generally decellularized in order to render them non-immunogenic, a process that needs to also retain some function of key proteins, such as some growth factors. Specific procedural steps are further detailed in the patents referenced above.

Examples of a typical epithelium having a basement membrane include tissues that have an epithelium such as the skin, intestine, urinary bladder, esophagus, stomach, cornea, and liver. The epithelial basement membrane may be in the form of a thin sheet of extracellular material contiguous with the basilar aspect of epithelial cells. Sheets of aggregated epithelial cells of similar type form an epithelium. Epithelial cells and their associated epithelial basement membrane may be positioned on the luminal portion of the tunica mucosa and constitute the internal surface of tubular and hollow organs and tissues of the body. Connective tissues and the submucosa, for example, are positioned on the abluminal or deep side of the basement membrane, and can include for example the submucosa of the intestine (SIS) and urinary bladder (UBS), and the dermis and subcutaneous tissues of the skin. Typically the material is rinsed with saline and optionally stored in a frozen hydrated state until used.

In addition to employing sheet ECMs to form the articles of the present invention, the ECM material may be fluidized or emulsified. Fluidized UBS, for example, can be prepared in a manner similar to the preparation of fluidized intestinal submucosa, as described in U.S. Pat. No. 5,275,826. The UBS is comminuted by tearing, cutting, grinding, shearing or the like. Grinding the UBS in a frozen or freeze-dried state is preferred although good results can be obtained as well by subjecting a suspension of submucosa pieces to treatment in a high speed (high shear) blender and dewatering, if necessary, by centrifuging and decanting excess water. Additionally, the comminuted fluidized tissue can be solubilized by enzymatic digestion of the bladder submucosa with a protease, such as trypsin or pepsin, or other appropriate enzymes for a period of time sufficient to solubilize said tissue and form a substantially homogeneous solution.

Other examples of ECM material suitable for use with the present invention include but are not limited to dermal extracellular matrix material, subcutaneous extracellular matrix material, large intestine extracellular matrix material, placental extracellular matrix material, ornamentum extracellular matrix material, heart extracellular matrix material, and lung extracellular matrix material, may be used, derived and preserved similarly as described herein for the SIS, SS, LBM, and UBM materials. Other organ tissue sources of basement membrane for use in accordance with this invention include spleen, lymph nodes, salivary glands, prostate, pancreas and other secreting glands. In general, any tissue of a mammal that has an extracellular matrix can be used for developing an extracellular matrix component of the invention.

Other tissues such as the liver and pancreas have a basement membrane that does not demonstrate the kind of tensile strength of the tissues defined as submucosa. However, other useful properties may be opportunistically employed from the extracellular matrices of such tissues as the liver, pancreas, placenta and lung tissues which have either basement membrane for extracellular matrix or interstitial membrane (as with the lung). These softer matrices support cells such as those in the organs from which the matrices are derived. Thus, certain benefits are to be found in using the extracellular matrices of these tissues, especially in combination with other such matrices like SIS and SS that may be stronger and which offer their particular advantages. Accordingly, any of these mammalian matrices can be used with potential effectiveness in the breast. Accordingly, the liver, lung, and pancreatic extracellular matrices may be quite suitable for generating some of the sheets, strips or pieces of the articles of the invention, or particulates or gels and may be used as such.

The article of extracellular matrix can comprise extracellular matrix combinations from such sources as, for example but not limited to, small intestine submucosa, liver basement membrane, stomach submucosa, urinary bladder submucosa, placental basement membrane, pancreatic basement membrane, large intestine submucosa, lung interstitial membrane, respiratory tract submucosa, heart extracellular matrix, dermal matrix, and in general extracellular matrix from any mammalian fetal tissue. Any one of these tissue sources can provide extracellular matrix that can then be manipulated into a designated form (e.g. sheet, strip or piece) for use in the articles of the invention, or particulate or emulsion or gel for the compositions of the invention.

The articles of the invention that are made of sheets, strips, or pieces of extracellular matrix can be made from a single source of extracellular matrix. The composition can also be made from two or more extracellular matrices isolated from a donor mammal or from a particular tissue source in that donor or multiple donors. In any event, the key factor is that at least two tissue sources from which the composition comprising mammalian extracellular matrix can be derived to form the composition derived from different tissue sources.

These tissue sources can be from the same mammal (for example the same cow, the same pig, the same rodent, the same human, etc.), the same species of mammal (e.g. a cow, a pig, a rodent, a human), or different mammalian animals (but the same species, e.g. cow 1 and cow 2, or pig 1 and pig 2), or different species of mammals (for example liver matrix from a pig, and small intestine submucosa from a cow, and urinary bladder submucosa from a dog).

The compositions of the invention are particulate ECM or emulsion ECM or gel ECM. The particulate is formed from lyophilized sheets of matrix that dry to form a powder. The powder can be sprinkled or dusted into the breast to form a new matrix in the space left after the lumpectomy procedure. The emulsion is formed from reconstituting the powder in a buffer such as saline, preferably a biologically safe buffer. Gels are formed of more concentrated emulsions, thus using less buffer and making a stiffer paste-type mixture.

Delivery of the sheets, particulate or emulsion or gel to the breast occurs after the tumor and surrounding tissue is removed. With regard to the sheets, the rice paper-like sheets can be crumpled into balls and placed in the space in the breast. Preferably the space is filled up with these balls which when they become wet will swell and expand within the breast. The particulate is light and can fly away easily so that care needs to be taken when placing the particulate in the breast that it actually arrives in the cavity. A broad syringe, or other delivery tool would probably be useful to accomplish delivery of the particulate. Generally it is optimal to fill the breast as much as possible with the particulate. The emulsion can be delivered to the breast using a syringe or catheter. The gel can likewise be delivered, provided the gel is soft enough to permit traveling through a syringe or catheter opening.

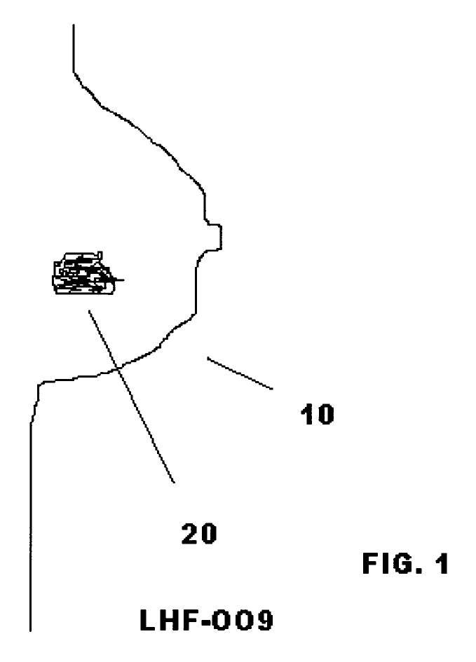

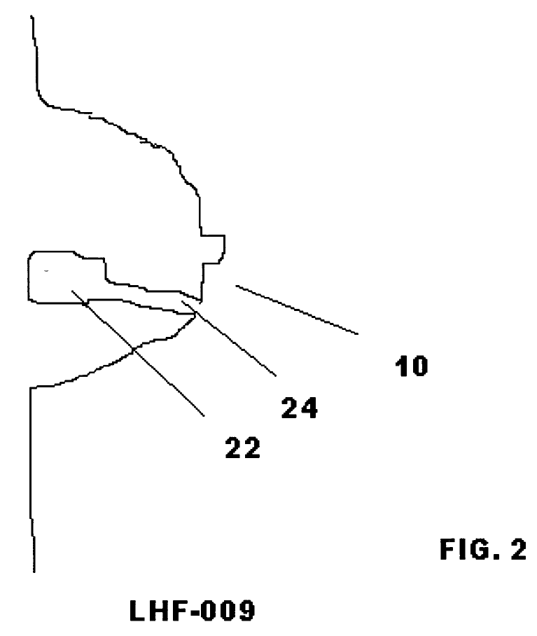

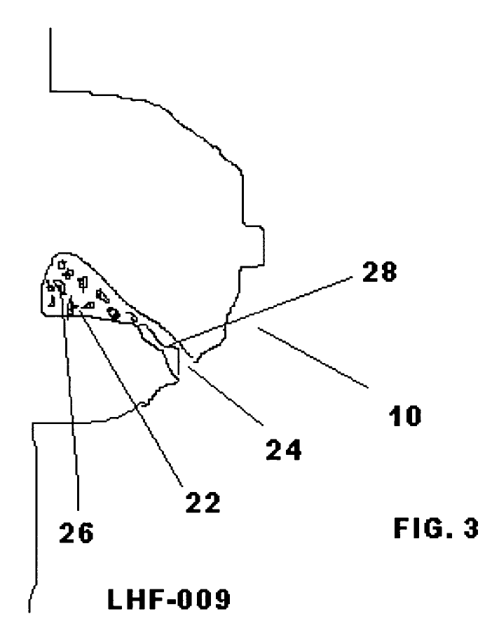

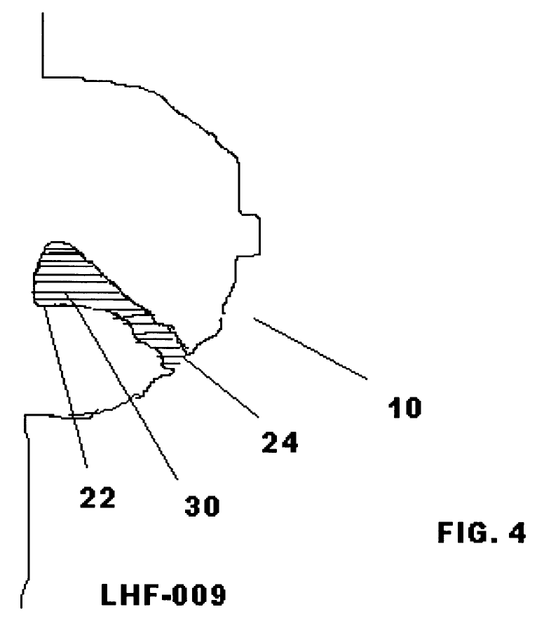

Turning now to the Figures to particularly illustrate the methods of the invention, FIG. 1 depicts a cross sectional view of a breast 10 having a lump 20. Often such lumps in breasts can be detected and visualized by magnetic resonance imaging (MRI). FIG. 2 depicts a cross sectional view of a breast after the tumor and surrounding tissue have been surgically removed in a lumpectomy procedure. The breast 10 depicts space 22 that remains after the lumpectomy procedure, and tissue tract 24 that is used to access the area having the tumor in the breast. FIG. 3 depicts breast 10 having a space 22, and a tissue tract 24, wherein the space 22 is filled with small wads or balls of crumpled ECM, 26. Tissue tract 24 is filled with a sheet of matrix 28. FIG. 4 depicts a cross sectional view of a breast 10, having space 22 and tissue tract 24 filled with ECM as an emulsion, gel or particulate 30. The method is practiced by finally surgically closing the opening at the surface of the breast of tissue tract 24.

All references cited are incorporated in their entirety. Although the foregoing invention has been described in detail for purposes of clarity of understanding, it will be obvious that certain modifications may be practiced within the scope of the appended claims.

What is claimed is:

1. An article or composition for placing at a site in a breast after tumor excision comprising mammalian extracellular matrix (ECM).

2. The article or composition of claim 1, wherein said mammalian ECM is small intestine submucosa.

3. The article or composition of claim 1, wherein said mammalian ECM is liver basement membrane.

4. The article or composition of claim 1, wherein said mammalian ECM is urinary bladder submucosa.

5. The article or composition of claim 1, wherein said mammalian ECM is stomach submucosa.

6. The article or composition of claim 1, wherein said mammalian ECM is porcine.

7. The article or composition of claim 1, wherein said ECM is in a single sheet form.

8. The article or composition of claim 1, wherein said ECM is a particulate.

9. The article or composition of claim 1, wherein said ECM is an emulsion or gel.

10. A method of regenerating excised tissue in a breast after a lumpectomy procedure comprising placing an article or composition comprising mammalian extracellular matrix (ECM) at a site of surgical excision of a tumor from said breast, and closing said surgical excision.

11. The method of claim 10, wherein said ECM is porcine.

12. The method of claim 10, wherein said ECM is selected from the group consisting of small intestine submucosa, stomach submucosa, liver basement membrane, and urinary bladder submucosa.

13. A method of reducing a likelihood of local tumor recurrence in a breast after a lumpectomy procedure comprising placing an article or composition comprising mammalian extracellular matrix (ECM) at a site of surgical excision of a tumor from said breast, and closing said surgical excision.

14. The method of claim 13, wherein said ECM is porcine.

15. The method of claim 13, wherein said ECM is selected from the group consisting of small intestine submucosa, stomach submucosa, liver basement membrane, and urinary bladder submucosa.

16. A method of reducing scar tissue formation in a breast after a lumpectomy procedure comprising placing an article or composition comprising mammalian extracellular matrix (ECM) at a site of surgical excision of a tumor from said breast, and closing said surgical excision.

17. The method of claim 16, wherein said ECM is porcine.

18. The method of claim 16, wherein said ECM is selected from the group consisting of small intestine submucosa, stomach submucosa, liver basement membrane, and urinary bladder submucosa.
